(19) United States
(12) Patent Application Publication
(10) Pub. No.: US 2006/0039882 A1
(43) Pub. Date: Feb. 23, 2006

(54) Hair care agents containing pregelatinized, cross-linked starch derivatives

(76) Inventors: Michael Demitz, Hamburg (DE); Horst Argembeaux, Wentorf (DE); Stephan Ruppert, Hamburg (DE); Ines Wallendszus, Hamburg (DE)

Correspondence Address: ALSTON & BIRD LLP;BANK OF AMERICA PLAZA, 101 SOUTH TRYON STREET, SUITE 4000, CHARLOTTE, NC, 28280-4000, US

(21) Appl. No.: 10/511,122

(22) PCT Filed: Apr. 9, 2003
(86) PCT No.: PCT/EP03/03677

§ 371 Date: Jun. 20, 2005

(30) Foreign Application Priority Data
Apr. 11, 2002 (DE) 102 16 500.9

(51) Int. Cl. A61K 7/06; A61K 7/11

(52) U.S. Cl. 424/70.13

(57) ABSTRACT

The invention relates to cosmetic hair care agents that contain either one or more pregelatinized, cross-linked starch derivatives and cationic polymers or one or more pregelatinized, cross-linked starch derivatives and non-ionic, amphoteric and/or anionic polymers.

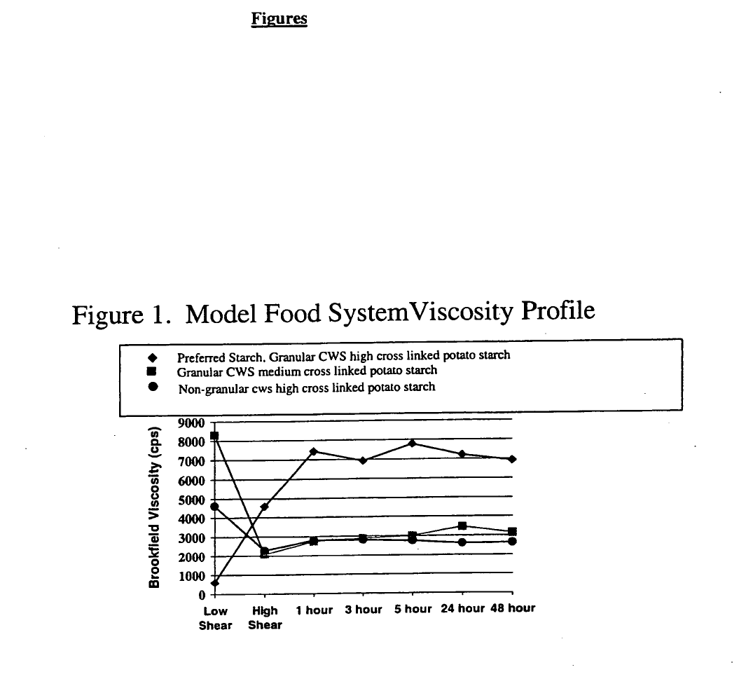

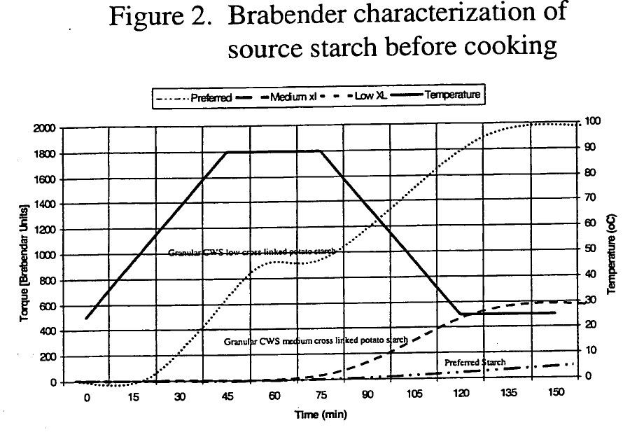

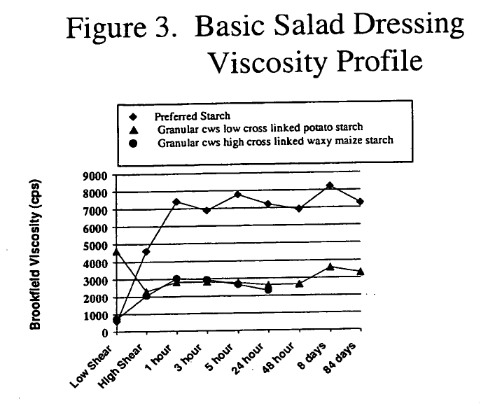

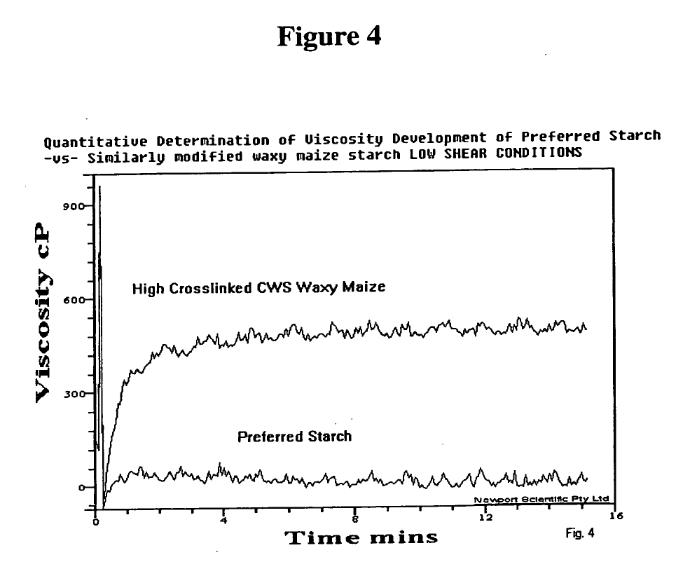

The present invention relates to cosmetic haircare agents containing polymers. The present invention relates in particular to haircare preparations or haircare agents containing substances and having especially improved combability and improved feel.

The entire human body, with the exception of the lips, the palms of the hands and the soles of the feet, is covered in hair, for the large part, however, with barely visible down. Because of the many nerve endings at the hair root, hair reacts sensitively to external influences such as wind or touch and is therefore a component of the sense of touch that should not be underestimated. The most important function of human head hair must, however, nowadays consist in helping to create the appearance of the person in a characteristic manner. Similarly to the skin, it fulfils a social function since, via its outward appearance, it contributes considerably to interpersonal relations and to the self-esteem of the individual.

The hair consists of the hair shaft which protrudes freely from the skin—the keratinized (dead) section which represents the actual visible hair—and the hair root which sticks in the skin—the living section, in which the visible hair is continually renewed. The hair shaft in turn is made up of three layers: a central section—the so-called hair marrow (medulla), which, however, in humans has retrogressed and is often missing altogether—also the marrow (cortex) and the external, horny layer up to ten layers thick (cuticle), which surrounds the entire hair.

Provided there are no pathological changes, it is virtually impossible to improve upon human hair in its freshly grown state. The section of a hair in the vicinity of the scalp accordingly has a virtually closed horny layer. In particular, the horny layer, being the external sheath of the hair, but also the inner region below the cuticle, are exposed to particular stress by environmental influences.

Significant effects on the loss of quality of a hair during its ageing are the effect of sunlight, mechanical stresses as a result of intensive combing or brushing, but also hair treatments, such as hair colorings and in particular bleachings, and hair shapings, for example permanent waving processes. Accordingly, oxidative stresses in particular often lead to hair damage.

Both UV-A and also UV-B radiation have a harmful effect on the hair, which is evident, for example, from the fact that certain amino acids, such as cystine and methionine are degraded or sulfur-sulfur bonds of keratin are cleaved, which in the worst case scenario can result in destruction of the hair. In addition, hair and scalp represent parts of the body which, due to their position, are subjected to a considerable amount of UV radiation when outdoors.

One aim of hair care is to maintain the natural condition of freshly grown hair over the longest period possible and, if it is lost, to restore it. Silky sheen, low porosity and a pleasant smooth feel are features of natural healthy hair.

Since the end of the previous century products for hair care have been developed specifically. This led to a large number of preparations both for general hair care and also for alleviating the anomalies of hair and of the scalp. In general, use is nowadays made of hair care cosmetics which are, after they have worked, either intended to be washed out of the hair again, or which should remain on the hair. The latter can be formulated such that they not only serve to care for the individual hair, but also improve the appearance of a hair style overall. Hair cared for in such a way is characterized by a pleasant feel, natural shine, increased body, suppleness and thus good stylability and strength and thus good hairstyle sit.

Products which serve exclusively to care for the hair are generally referred to as hair conditioning agents or conditioners. After a longer or shorter residence time on the hair, these can be rinsed out (rinse-off products, e.g. rinses, hair treatments) or they remain following application to the hair (leave-on products). The products can have various consistencies, meaning that they can be applied in very different ways. They may be emulsions or gels or low-viscosity solutions which are applied, for example, by means of spray applications, or foams, which are generated, for example, by suitable pressurized gas packings or special foam pumps during application. Creamy, opaque and clearly transparent products can be found on the market.

Depending on the intended use, quite diverse active ingredients or combinations of active ingredients are found in such conditioners. Some which serve more to protect the hair, such as antioxidants or UV filters, others which make the hair supple, such as, for example, cationic surfactants. Achieving ever greater importance are polymeric active ingredients which have very diverse properties depending on their nature, molecular weight and charge. However, an improvement in the nature of the surface of the hair is clearly at the forefront.

Despite a great diversity of polymers which are available to the person skilled in the art, some disadvantages which are associated with the use of polymers have still not been completely overcome. For example, polymers which give the hair a certain strength and thus volume often exhibit a poor feel sensation and poor combability; polymers which make the hair supple often lead to it being weighed down, which is associated with inadequate volume. The use of starch derivatives has in the past also been unable to overcome the described disadvantages of the prior art.

It has been found, in a manner which is surprising and was unforeseeable by the person skilled in the art, that cosmetic haircare agents either containing one or more pregelatinized crosslinked starch derivatives and cationic polymers or containing one or more pregelatinized, crosslinked starch derivatives and nonionic, amphoteric and/or anionic polymers overcome the disadvantages of the prior art. By using the polymer combinations according to the invention, hair-treatment compositions with excellent care performances are obtained, where the combination of starch derivatives with cationic polymers improves the suppleness-improving care properties in a synergistic manner without weighing down the hair. The combination of starch derivatives with nonionic, amphoteric and anionic polymers likewise synergistically improves the setting properties, which is associated with better hair volume. The hair is likewise given a pleasant feel sensation and can be styled easily.

It is preferred here if the pregelatinized, crosslinked starch derivatives used are hydroxypropylated starch phosphate esters, particularly preferably hydroxypropyl distarch phosphates.

It is preferred if the cationic polymers used are polymeric quaternized ammonium salts of hydroxyethylcellulose which have been modified with a trimethylammonium-substituted epoxide, depolymerized and subsequently quaternized guar gum derivatives and/or quaternized guar derivatives.

It is also preferred if the cationic polymers chosen are at least one from the group of cationic cellulose derivatives.

It is further preferred if the nonionic, amphoteric and/or anionic polymers used are PVP/VA copolymers, anionic acrylate copolymers and/or amphoteric amide/acrylate/methacrylate copolymers, particularly preferably PVP/VA copolymers. The content of pregelatinized, crosslinked starch derivatives is particularly preferably 20 to 99.9% by weight, very particularly preferably 25 to 95% by weight, based on the total content of polymers.

The total content of polymers is preferably 0.1 to 3% by weight, particularly preferably 0.2 to 1.5% by weight, based on the total weight of the preparation. It is further preferred if further surfactants and/or cosmetic or dermatological auxiliaries, additives and/or active ingredients are additionally present.

Although U.S. Pat. No. 6,248,338 describes cosmetic preparations with pregelatinized, crosslinked starch derivatives, this specification was unable to point the way to the present invention.

According to the invention, it is advantageous if the pregelatinized, crosslinked starch derivatives used are hydroxypropylated phosphate esters. Of particular advantage are those starch derivatives as described in U.S. Pat. No. 6,248,338, particularly advantageously hydroxypropyl distarch phosphate. Very particular preference here is given to the use of a hydroxypropyl distarch phosphate, as is sold in the form of the product Structure® XL by National Starch.

The combination of starch derivatives (Structure XL) with a polymeric quaternized ammonium salt of hydroxyethylcellulose which is modified with a trimethylammonium-substituted epoxide (INCI: Polyquaternium-10) (Ucare Polymer JR 400 from Amerchol) or with a depolymerized guar gum derivative which has been quaternized (INCI: Guar Hydroxypropyl Trimonium Chloride) (Jaguar Excel from Rhodia) or with a quaternized guar derivative (Cosmedia Guar C 261 (Cognis)) or with a nonionic PVP/VA copolymer (Luviskol VA 64W, BASF) or with an anionic acrylate copolymer (Luviflex soft, BASF) or with an amphoteric amide/acrylate/methacrylate copolymer (Amphomer, National Starch) has proven to be particularly preferred.

According to the invention, it is advantageous if the pregelatinized, crosslinked starch derivatives used are hydroxypropylated phosphate esters. Of particular advantage are those starch derivatives as described in U.S. Pat. No. 6,248,338, particularly advantageously hydroxypropyl distarch phosphate. Very particular preference here is given to the use of a hydroxypropyl distarch phosphate, as is sold in the form of the product Structure® XL by National Starch.

Cosmetic and dermatological preparations according to the invention may be present in various forms. Thus, for example, they may be a solution, an anhydrous preparation, an emulsion or microemulsion of the water-in-oil (W/O) type or of the oil-in-water (O/W) type, a multiple emulsion, for example of the water-in-oil-in-water (W/O/W) type, a gel, a solid stick, an ointment or else an aerosol.

The haircare agents are topical preparations. They can have the customary composition and be used for the treatment and the care of the scalp and/or the hair or as a photoprotective preparation. For use, the preparations according to the invention are applied to the scalp and the hair in an adequate amount in the manner customary for cosmetics and haircare agents.

Advantageously, preparations for the purposes of the present invention may be in the form of a hair treatment or hair rinse.

The compositions according to the invention can, for example, be in the form of preparations which are sprayable from aerosol containers, squeezable bottles or via a pump, spray or foaming device, but also in the form of a composition which can be applied from normal bottles and containers.

For the purposes of the present invention, suitable propellants for cosmetic or dermatological preparations which are sprayable from aerosol containers are the customary known readily volatile liquefied propellants, for example dimethyl ether, hydrocarbons (propane, butane, isobutane), which can be used on their own or in a mixture with one another. Compressed air, nitrogen, nitrogen dioxide or carbon dioxide or mixtures of these substances are also used advantageously.

The person skilled in the art of course is aware that there are propellant gases which are nontoxic per se which would in principle be suitable for realizing the present invention in the form of aerosol preparations, but which nevertheless have to be disregarded due to an unacceptable impact on the environment or other accompanying circumstances, in particular fluorocarbons and chlorofluorocarbons (CFCs).

Besides an effective content of active ingredients according to the invention, preparations according to the invention advantageously also comprise customary active substances, ingredients, additives and/or auxiliaries.

A large number of products is referred to as haircare agents, the most important representatives of which are pretreatment agents, hair tonics and hair treatment agents.

The basic substances of haircare agents are, for example, fatty alcohols, waxes, paraffins, Vaseline, paraffin oil and solvents.

Fatty alcohols are, for example, straight- or branched-chain aliphatic monohydric alcohols having 6-22 carbon atoms in the molecule. In cosmetics, preference is given to using straight-chain fatty alcohols with a chain length of 12-18 carbon atoms. These fatty alcohols are soft, colorless masses, virtually nontoxic and well tolerated by the skin. Fatty alcohols are preferably used for the preparation of hair treatments and styling creams, with cetyl alcohol and stearyl alcohol being of particular importance.

Waxes are fatty acid esters which occur in animal and vegetable products, but can also be prepared synthetically. Probably the best known naturally occurring wax is beeswax which comprises cerin and myricin as main constituents. However, wax is a generic term for a number of natural and synthetic substances which as a rule represent semisolid, white, odorless and water-insoluble masses.

Paraffins in the cosmetics sense are white, odorless masses of straight-chain high molecular weight hydrocarbons. Due to their properties comparable with those of waxes, they are also often referred to as petroleum waxes.

Vaseline is a mixture of branched-chain paraffins with a low content of cyclic paraffins. It is a soft, transparent and water-insoluble mass with a low intrinsic odor and is produced during the treatment of petroleum.

Paraffin oil is a mixture of saturated liquid hydrocarbons. It is insoluble in water, but miscible with fatty alcohols and waxes. It is used as additive in haircare agents for regulating the consistency.

Solvents play a considerable role in cosmetics. Of the large number of solvents which are available, ethanol is of most importance. It is used for preparing hair tonics where, due to its disinfecting properties, it fulfills the function of an active ingredient at the same time.

Auxiliaries can be used in order to improve certain properties of the haircare agents, e.g. consistency, thermal stability and photo stability, appearance and odor, and to facilitate their preparation. For example, the following are added as required:
Emulsifiers in order to reduce the interfacial tension between two per se immiscible phases until their fine mixing becomes possible,
Thickeners in order to increase the stability of emulsions and to adjust their viscosity,
UV absorbers in order to improve the photostability of the dyes and other photosensitive components present in the haircare agents. They also serve to protect the hair against the effects of light.
Preservatives in order to prevent microbial decomposition.
Antioxidants in order to prevent changes in odor which can be caused by oxidation processes.
Dyes in order to give the haircare agents a pleasing appearance.
Perfume oils in order to give haircare agents a pleasant scent and to conceal secondary odors of the raw materials.

The amount of the basic substances is, for example, 85 to 99.999% by weight, preferably 90 to 99.99% by weight, in each case based on the total weight of the preparation.

Quaternary ammonium compounds are an important group of the special active ingredients which are used to produce haircare agents. Haircare agents, in particular hair treatments, are given essential properties such as improvement of combability, and feel and prevention of static charging of the hair primarily by using quaternary ammonium compounds.

The properties of quaternary ammonium compounds are determined by the cationic group on the one hand and by the type of lipophilic radicals of this group on the other hand. The compounds in which one to two radicals of longer-chain alkyl groups, such as lauryl, cetyl or stearyl groups, and the remaining radicals are methyl groups are suitable. Products of this type are preferably used as chlorides, bromides and methosulfates.

Also suitable are polymeric quaternary ammonium compounds, macromolecules whose essential feature is the presence of two or more quaternary ammonium groups in the molecule. As a result, their ability to adhere to the hair is significantly increased.

Cationic surfactants to be used particularly advantageously are

1. Alkylamines

2. Alkylimidazoles

3. Ethoxylated amines and

4. Quaternary surfactants.

5. Ester quats

Quaternary surfactants contain at least one N atom which is covalently bonded to 4 alkyl or aryl groups. Irrespective of the pH, this leads to a positive charge. Alkylbetaine, alkylamidopropylbetaine and alkylamidopropylhydroxysultaine are advantageous. The cationic surfactants used according to the invention can also preferably be chosen from the group of quaternary ammonium compounds, in particular benzyltrialkylammonium chloride, and also alkyltrialkylammonium salts, for example cetyltrimethylammonium chloride or bromide, alkyldimethylhydroxyethylammonium chlorides or bromides, dialkyldimethylammonium chlorides or bromides, alkylamidoethyltriethylammonium ether sulfates, alkylpyridinium salts, for example lauryl- or cetylpyrimidinium chloride, imidazoline derivatives and compounds with cationic character, such as amine oxides, for example alkyldimethylamine oxides or alkylaminoethyldimethylamine oxides. Cetyltrimethylammonium salts in particular are to be used advantageously.

Monomeric or polymeric quaternary ammonium compounds are used widely in hair rinses and hair treatments, e.g. in concentrations of 0.5-5% by weight. These include cetrimonium chloride, as is supplied under the name Dehyquart A by Henkel, or distearoylethyl hydroxethylmonium methosulfate, as is supplied under the name Dehyquart F 75 by Henkel.

If the cosmetic or dermatological preparations are in the form of a lotion which is rinsed out and which is applied, for example, before or after bleaching, before or after shampooing, between two shampooing steps, before or after permanent-wave treatment, then they are, for example, emulsions which optionally comprise surface-active substances whose concentration can be between 0.1 and 10% by weight, preferably between 0.2 and 5% by weight.

A cosmetic preparation in the form of a lotion which is not rinsed out, in particular a lotion for arranging the hair, a lotion which is used while blow-drying the hair, a styling and treatment lotion, is generally an emulsion and comprises the combinations according to the invention. However, it is in some cases advantageous if the lotion according to the invention is in the form of a microemulsion or an aqueous or aqueous-alcoholic solution.

According to the invention, cosmetic preparations for the treatment and care of the hair can be in the form of gels which comprise organic thickeners, e.g. gum arabic, xanthan gum, sodium alginate, cellulose derivatives, preferably methylcellulose, hydroxymethylcellulose, hydroxyethylcellulose, hydroxypropylcellulose, hydroxypropylmethylcellulose or inorganic thickeners, e.g. aluminum silicates, such as, for example, bentonite, or a mixture of polyethylene glycol and polyethylene glycol stearate or distearate. The thickener is present in the gel, for example, in an amount between 0.1 and 30% by weight, preferably between 0.5 and 15% by weight.

The percentages given above refer to the total weight of the preparations.

The preparations according to the invention can comprise cosmetic auxiliaries as are customarily used in such preparations, e.g. preservatives, perfumes, substances for preventing foaming, foam stabilizers, dyes, pigments which have a coloring effect, thickeners, surface-active substances, emulsifiers, softening, moisturizing and/or humectant substances, refatting agents, fats, oils, waxes, alcohols, polyols and their toxicological compatible ethers and esters, branched and/or unbranched hydrocarbons, further antioxidants, stabilizers, pH regulators, bodying agents, bactericides, deodorants, antimicrobial substances, antistats, UV absorbers, complexing and sequestering agents, pearlizing agents, polymers, electrolytes, organic solvents, silicone derivatives, plant extracts, vitamins and/or other active ingredients or other customary constituents of a cosmetic or dermatological formulation. Solubility promoters, e.g. for incorporating hydrophobic components, such as, for example, perfume preparations, may also be present.

The total amount of the auxiliaries is, for example, 0.001 to 15% by weight, preferably 0.01 to 10% by weight, in each case based on the total weight of the preparation.

The amount of thickeners is, for example, 0.05 to 5.0% by weight, preferably 0.1 to 3.0% by weight, in particular 0.15 to 2.0% by weight, in each case based on the total weight of the preparation.

The water content of the preparations is, for example, 60 to 95% by weight, preferably 75 to 95% by weight, in particular 80 to 90% by weight, in each case based on the total weight of the preparation.

Antioxidants which may additionally be used according to the invention are all antioxidants customary or suitable for cosmetic and/or dermatological applications.

The total amount of the antioxidants is, for example, 0.000.001 to 2% by weight, preferably 0.001 to 1% by weight, in each case based on the total weight of the preparation.

Further antioxidants are advantageously chosen from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximines, homocysteine sulfoximine, buthionine sulfones, penta-, hexa-, heptathionine sulfoximine) in very low tolerated doses (e.g. pmol to μmol/kg), also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin resin, rutinic acid and derivatives thereof, α-glycosylrutin, ferulic acid, furfurylideneglucitol, carnosine, butylhydroxytoluene, butylhydroxyanisol, nordihydroguaiacic acid, nordihydroguaiaretic acid, trihydroxybutyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of the specified active ingredients which are suitable according to the invention.

Preparations according to the invention may advantageously also comprise substances which absorb UV radiation in the UV-B region, where the total amount of the filter substances is, for example, 0.001% by weight to 30% by weight, preferably 0.05 to 10% by weight, in particular 0.1 to 1.0% by weight, based on the total weight of the preparations, in order to provide cosmetic preparations which protect the hair and/or the skin from the entire range of ultraviolet radiation. They can also serve as sunscreens for the hair or the skin, in particular the scalp.

If the emulsions according to the invention comprise UV-B filter substances, these may advantageously be water-soluble. Advantageous water-soluble UV-B filters are, for example:
salts of 2-phenylbenzimidazole-5-sulfonic acid, such as its sodium, potassium or its triethanolammonium salt, and the 2-phenylbenzimidazole-5-sulfonic acid itself;
sulfonic acid derivatives of benzophenones, preferably 2-hydroxy-4-methoxybenzophenone-5-sulfonic acid and its salts;
sulfonic acid derivatives of 3-benzylidenecamphor, such as, for example, 4-(2-oxo-3-bornylidenemethyl)benzenesulfonic acid, 2-methyl-5-(2-oxo-3-bornylidene-methyl)sulfonic acid and its salts.

It may also be advantageous to admix preparations according to the invention with UV-A filters which are hitherto customarily present in cosmetic preparations. It is possible to use the amounts used for the UV-B combination.

The preparations according to the invention can be prepared in the customary manner by mixing the individual constituents. The active ingredients of the combinations according to the invention or else the premixed constituents of the combinations according to the invention may be added in the mixing operation.

The pH of the preparations can be adjusted in a known manner by adding acids or bases, preferably by adding buffer mixtures, e.g. based on citric acid/citrate or phosphoric acid phosphate buffer mixtures. Preferably, the pH is below 10, e.g. in the range from 2-7, in particular in the range from 3-5.

Unless stated otherwise, all amounts, fractions and percentages are based on the weight and the total amount or on the total weight of the preparations or of the particular mixture.

The examples below illustrate the invention.

The amounts given in the examples are percentages by weight, based on the total weight of the particular preparation.

### EXAMPLES

### Examples 1-3

| Hair treatment | 1 | 2 | 3 |
|---|---|---|---|
| Hydroxypropylmethylcellulose | 0.5 | 0.5 | 0.5 |
| Cetrimonium bromide | 1.0 | 1.0 | 1.0 |
| Glycerol | 3.0 | 3.0 | 3.0 |
| Cetearyl alcohol | 2.5 | 2.5 | 2.5 |
| Glyceryl stearate | 2.0 | 2.0 | 2.0 |
| Hydroxypropyl starch phosphate ester | 0.2 | 0.5 | 0.8 |
| (Structure XL) | | | |
| Polyquaternium-10 | 0.1 | — | — |
| Guar hydroxypropyl trimonium | — | 0.2 | — |
| chloride | | | |
| PVP/VA copolymer | — | — | 0.1 |
| Preservative, perfume, pH adjustment | q.s. | q.s. | q.s. |
| Water, demin. (demineralized) | ad 100.0 | ad 100.0 | ad 100.0 |

The pH is adjusted to 3.5.

### Examples 4-6

| Hair rinses | 4 | 5 | 6 |
|---|---|---|---|
| Behentrimonium chloride | 1.0 | 1.0 | 1.0 |
| Glycerol | 3.0 | 3.0 | 3.0 |
| Hydroxyethylcellulose | 0.2 | 0.2 | 0.2 |
| Cetearyl alcohol | 3.0 | 3.0 | 3.0 |
| Hydroxypropyl starch phosphate ester | 0.2 | 0.5 | 0.8 |
| (Structure XL) | | | |
| Polyquaternium-10 | 0.1 | — | — |
| Guar hydroxypropyl trimonium | — | 0.2 | — |
| chloride | | | |
| PVP/VA copolymer | — | — | 0.1 |
| Preservative, perfume, pH adjustment | q.s. | q.s. | q.s. |
| Water, demin. | ad 100.0 | ad 100.0 | ad 100.0 |

The pH is adjusted to 3.0.

### Examples 7-9

| Spray conditioners | 7 | 8 | 9 |
|---|---|---|---|
| Benzophenone-4 | 0.05 | 0.03 | 0.04 |
| Hydrolyzed keratin | 0.5 | 1.0 | 0.2 |
| Polyquaternium-10 | 0.5 | — | 1.0 |
| PVP/VA copolymer | — | 0.7 | — |
| Hydroxypropyl starch phosphate ester | 0.2 | 0.5 | 0.8 |
| (Structure XL) | | | |
| Cetrimonium chloride | 0.2 | 0.8 | 0.1 |
| Preservative, perfume, pH adjustment | q.s. | q.s. | q.s. |
| Water, demin. | ad 100.0 | ad 100.0 | ad 100.0 |

The pH is adjusted to 5.5.

### Examples 10-12

| Leave-on conditioner | 10 | 11 | 12 |
|---|---|---|---|
| Cetyl alcohol | 1.5 | 1.8 | 2.0 |
| C12-13 alkyl lactate | 2.0 | 1.0 | 1.5 |
| Cetrimonium chloride | 0.3 | 0.2 | 0.2 |
| Acrylates/C10-30 alkyl acrylates | 0.5 | 0.3 | 0.2 |
| crosspolymer | | | |
| Polyquaternium-10 | — | 0.2 | 0.5 |
| PVP/VA copolymer | 0.4 | — | — |
| Hydroxypropyl starch phosphate ester | 0.2 | 0.5 | 0.8 |
| (Structure XL) | | | |
| Preservative, perfume, pH adjustment | q.s. | q.s. | q.s. |
| Water, demin. | ad 100.0 | ad 100.0 | ad 100.0 |

1. A cosmetic hair care agent comprising one or more pregelatinized, crosslinked starch derivatives and one or more polymers selected from the group consisting of cationic polymers and nonionic, amphoteric, or anionic polymers.

2. The hair care agent as claimed in claim 1, wherein the one or more pregelatinized, crosslinked starch derivatives include a hydroxypropylated starch phosphate esters.

3. The hair care agent as claimed in claim 1, wherein the one or more pregelatinized, crosslinked starch derivatives include a hydroxypropyl distarch phosphates.

4. The hair care agent as claimed in claim 1, wherein the cationic polymers are selected from the group consisting of polymeric quaternized ammonium salts of hydroxyethylcellulose which has been modified with a trimethylammonium-substituted epoxide, depolymerized and subsequently quaternized guar gum derivatives, and quaternized guar derivatives.

5. The hair care agent as claimed in claim 1, wherein cationic polymers include at least one cationic cellulose derivatives.

6. The hair care agent as claimed in claim 1, wherein the nonionic, amphoteric, or anionic polymers are selected from the group consisting of PVP/VA copolymers, anionic acrylate copolymers, and amphoteric amide/acrylate/methacrylate copolymers.

7. The hair care agent as claimed in claim 6, wherein the nonionic, amphoteric, or anionic polymers include at least one of PVP/VA copolymers.

8. The hair care agent as claimed in claim 1, further comprising one or more surfactants, wherein the surfactants are selected from the group consisting of fatty alcohols having 6 to 22 carbon atoms and alkylamidopropylbetaine.

9. The hair care agent as claimed in claim 1, wherein the total content of polymers is 0.1 to 3% by weight, based on the total weight of the haircare agent.

10. The hair care agent as claimed in claim 1, further comprising one or more additional components selected from the group consisting of surfactants, cosmetic or dermatological auxiliaries, additives, and active ingredients.

11. The hair care agent as claimed in claim 8, wherein the one or more surfactants are selected from the group consisting of sodium myristyl ether sulfate, sodium lauryl ether sulfate, cocoamidoproylbetaine, disodium PEG-5 lauryl citrate sulfosuccinate, and sodium cocoamphoacetate.

12. The hair care agent as claimed in claim 9, wherein the total content of polymers is 0.2 to 1.5% by weight based on the total weight of the haircare agent.

13. The hair care agent as claimed in claim 1, wherein the content of pregelatinized, crosslinked starch derivatives is 20 to 99.9% by weight based on the total content of polymers.

14. A cosmetic or dermatological preparation comprising a hair care agent as claimed in claim 1.

15. A cosmetic hair care agent comprising one or more pregelatinized, crosslinked starch derivatives and one or more polymeric materials selected from the group consisting of:
quaternized ammonium salts of hydroxyethylcellulose which is modified with a trimethylammonium-substituted epoxide;
depolymerized guar gum derivatives which have been quaternized;
quaternized guar gum derivatives;
nonionic PVP/VA copolymers;
anionic acrylate copolymers; and
amphoteric amide/acrylate/methacrylate copolymers.

16. The hair care agent as claimed in claim 15, wherein the one or more pregelatinized, crosslinked starch derivatives include a hydroxypropylated starch phosphate ester.

17. The hair care agent as claimed in claim 16, wherein the one or more pregelatinized, crosslinked starch derivatives include hydroxypropyl distarch phosphate.
